SEQUENCE LISTING (22 sequences)

SEQ ID NO: 1 - DNA, 13 bp, Artificial Sequence
  Fully synthetic
ctcctgcagtgcc

SEQ ID NO: 2 - DNA, 16 bp, Artificial Sequence
  Fully synthetic
gcaggtcgcggttctc

SEQ ID NO: 3 - DNA, 79 bp, Artificial Sequence
  Fully synthetic
ctcctgcagtgccccctcgcatcaataccgatcattcttcccctcgcatcaataccgatcatcgcaggtcgcggttctc

SEQ ID NO: 4 - DNA, 22 bp, Artificial Sequence
  Fully synthetic
ccctcgcatcaataccgatcat

SEQ ID NO: 5 - DNA, 535 bp, Artificial Sequence
  Fully synthetic
agtgcaatactgcccaagcccgggcggggtctctgttctctggcagaggaggtcccttggcagcgggaagcgccctctctttctctcgccgccgctccgagtctgcgccctggtgccaggcgctcagctcggcgctcccctgtgctcgcccggcgcccactcattcgcagcccggccttcgtcgccgccgcctccctgctgctcctcctcctttccccagcccgccgcggccatggcggacagcgccagcgagagcgacacggacggggcggggggcaacagcagcagctcggccgccatgcagtcgtcctgctcgtcgacctcgggcggcggcggtggcggcgggggaggcggcggcggtgggaagtcggggggcattgtcatctcgccgttccgcctggaggagctcaccaaccgcctggcctcgctgcagcaagagaacaaggtgctgaagatagagctggagacctacaaactgaagtgcaaggcactgcaggaggagaaccgcgacctgcgcaaagccagcgtgaccatc

SEQ ID NO: 6 - DNA, 13 bp, Artificial Sequence
  Fully synthetic
ggaaggccgttgc

SEQ ID NO: 7 - DNA, 21 bp, Artificial Sequence
  Fully synthetic
accctgctctgcctttcagat

SEQ ID NO: 8 - DNA, 84 bp, Artificial Sequence
  Fully synthetic
ggaaggccgttgcctgcgaatagccatccactccattcttctgcgaatagccatccactccataccctgctctgcctttcagat

SEQ ID NO: 9 - DNA, 22 bp, Artificial Sequence
  Fully synthetic
ccctcgcatcaataccgatcat

SEQ ID NO: 10 - DNA, 148 bp, Artificial Sequence
  Fully synthetic
gaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaag

SEQ ID NO: 11 - DNA, 80 bp, Artificial Sequence
  Fully synthetic
cctcctgcagtgcctccctcgcatcaataccgatcattcttcccctcgcatcaataccgatcatcgcaggtcgcggttct

SEQ ID NO: 12 - DNA, 81 bp, Artificial Sequence
  Fully synthetic
ctcctgcagtgccttccctcgcatcaataccgatcattcttcccctcgcatcaataccgatcatcgcaggtcgcggttctc

SEQ ID NO: 13 - DNA, 82 bp, Artificial Sequence
  Fully synthetic
tcctgcagtgccttgccctcgcatcaataccgatcattcttcccctcgcatcaataccgatcatcgcaggtcgcggttctcc

SEQ ID NO: 14 - DNA, 80 bp, Artificial Sequence
  Fully synthetic
ttctcctcctgcagtgcctccctcgcatcaataccgatcattcttcccctcgcatcaataccgatcatcgcaggtcgcgg

SEQ ID NO: 15 - DNA, 81 bp, Artificial Sequence
  Fully synthetic
tcctgcagtgccttccctcgcatcaataccgatcattcttcccctcgcatcaataccgatcatcgcaggtcgcggttctcc

SEQ ID NO: 16 - DNA, 80 bp, Artificial Sequence
  Fully synthetic
tcagatggaaggccgctgcgaatagccatccactccattcttctgcgaatagccatccactccataccctgctctgcctt

SEQ ID NO: 17 - DNA, 80 bp, Artificial Sequence
  Fully synthetic
gatggaaggccgctgcgaatagccatccactccattcttctgcgaatagccatccactccataccctgctctgcctttca

SEQ ID NO: 18 - DNA, 82 bp, Artificial Sequence
  Fully synthetic
gatggaaggccgttctgcgaatagccatccactccattcttctgcgaatagccatccactccataccctgctctgcctttca

SEQ ID NO: 19 - DNA, 81 bp, Artificial Sequence
  Fully synthetic
tttcagatggaaggccgtctgcgaatagccatccactccattcttctgcgaatagccatccactccataccctgctctgcc

SEQ ID NO: 20 - DNA, 82 bp, Artificial Sequence
  Fully synthetic
tggaaggccgttctgcgaatagccatccactccattcttctgcgaatagccatccactccataccctgctctgcctttcaga

SEQ ID NO: 21 - DNA, 10 bp, Artificial Sequence
  Fully synthetic
  misc_feature (4)..(7): n is a, c, g, or t
caynnnnrtg

SEQ ID NO: 22 - DNA, 10 bp, Artificial Sequence
  Fully synthetic
  misc_feature (4)..(7): n is a, c, g, or t
cacnnnngtg

(19) United States
(12) Patent Application Publication
(10) Pub. No.: US 2022/0056523 A1
(43) Pub. Date: Feb. 24, 2022

(54) PROBE, KIT COMPRISING THE PROBE, AND METHOD FOR IDENTIFYING CCDC6-RET FUSION GENE

(71) Applicant: Padlock (Tianjin) Biotechnology Ltd., Tianjin (CN)

(72) Inventors: Dan ZHAO, Tianjin (CN); Huan WANG, Tianjin (CN); Bo PENG, Tianjin (CN)

(21) Appl. No.: 17/337,398

(22) Filed: Jun. 2, 2021

(30) Foreign Application Priority Data
Aug. 18, 2020 (CN) 202010830940.9

(51) Int. Cl. C12Q 1/6876; C12Q 1/682

(52) U.S. Cl. CPC C12Q 1/6876; C12Q 1/682

(57) ABSTRACT

The disclosure provides a pair of probes for identifying CCDC6-RET fusion gene in a cell nucleus. The pair of probes includes a first probe for identifying a CCDC6 gene; and a second probe for identifying a RET gene.

### CROSS-REFERENCE TO RELATED APPLICATIONS

Pursuant to 35 U.S.C. § 119 and the Paris Convention Treaty, this application claims foreign priority to Chinese Patent Application No. 202010830940.9 filed Aug. 18, 2020, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of molecular biology and oncology, and more particularly, to a pair of probes, a kit comprising the probes, and a method for identifying CCDC6-RET fusion gene.

In recent years, with the rapid development of molecular pathological diagnosis, there has been an improvement in diagnosis and treatment of malignant tumors. The malignant tumors typically occur and develop as a result of inactivation of some tumor suppressor genes and activation of proto-oncogenes. RET proto-oncogene (RET gene) is located on the long arm of chromosome 10, about 80 kb in length, and contains 21 exons. RET gene encodes a protein with the capacity to induce malignant tumors when over-expressed within cells. RET gene in cancer cells can be mutated in a variety of ways, from point mutation, amplification, to gene rearrangement. Coiled-coil domain containing 6 gene (CCDC6 gene) encodes a protein containing a coiled-coil domain, is located on the long arm of chromosome 10, about 117 kb in length, and contains 9 exons. The expression products of the CCDC6-RET fusion gene cause the formation of papillary thyroid cancer. According to the data from Catalogue of Somatic Mutations in Cancer database (COSMIC database), about 12% of patient sample with papillary thyroid cancer studied are positive for the fusion gene.

Conventional detection method identifies the CCDC6-RET fusion gene through RNA extraction from tumor tissues, reverse transcription, and high-throughput sequencing or next-generation high-throughput sequencing. The detection method is complicated, time-consuming and cannot locate gene in the cell nucleus. In situ detection refers to the detection of DNA in the natural state in the cell nucleus, for example, a method of positioning DNA sequences within the nucleus, which is simple and convenient for observation. The current method of positioning CCDC6-RET fusion gene in the nucleus cannot use fluorescence in situ hybridization (FISH) probe to detect DNA sequences, because the fusion sequence on the genome is not known.

### SUMMARY

Accordingly, the disclosure provides a pair of probes for visually identifying CCDC6-RET fusion gene in cell nucleus.

The pair of probes comprise a first probe for identifying CCDC6 gene and a second probe for identifying RET gene.

Gene fusion can occur when RET gene is ectopically expressed in many tumor cells. The fusion gene encodes a fusion protein that is not expressed in the human body, leading to malignant proliferation of cancer cells and development of cancer. In general, RET gene is likely to fuse to CCDC6 gene. Sequencing of human genome indicates that CCDC6 gene and RET gene are located on the chromosome 10, with a physical distance of 17,925,554 bp, which indicates that the two genes are not clustered but rather scattered in the nucleus. Typically, CCDC6-RET fusion does not occur, and the first probe and the second probe bind to the corresponding fluorescent probes and produce two types of fluorescent spots that do not gather or approach to each other. While when CCDC6 gene and RET gene are fused together, the two types of fluorescent spots approach to one another to aggregate.

There is no specific limitation on the binding site of the first probe to CCDC6 gene, nor is there any specific limitation on the binding site of the second probe binding to RET gene. The sequences of the first probe and the second probe are designed according to the fusion site of the CCDC6-RET fusion gene.

The first probe is configured to identify the exon 1 of the CCDC6 gene, and/or the second probe is configured to identify the exon 12 of the RET gene.

The exon 1 of the CCDC6 gene is fused to the exon 12 of the RET gene, which is the more common type of the CCDC6-RET fusion gene. CCDC6 gene and RET gene are ectopically expressed in some thyroid cancer cells and lung cancer cells, thereby encoding a fusion protein in which the amino acids encoded by the exon 1 of the CCDC6 gene and the exon 12 of the RET gene are spliced together. The fusion protein aggregates to one another, which is very different from that in the normal cells. Therefore, the first probe and the second probe have the ability to accurately detect the presence of the CCDC6-RET fusion gene in the samples to be tested.

The first probe and the second probe each comprise: (1) a first region complementary to a 5′-end sequence of a gene of interest; (2) a second region complementary to a 3′-end sequence of the gene of interest; and (3) a third region which is a circular sequence complementary to a fluorescent probe and is located between the first region and the second region.

The gene of interest of the first probe refers to the exon 1 of human CCDC6 gene. The sequence of the exon 1 is shown in SEQ ID NO: 5:

5′-agtgcaatactgcccaagcccgggcggggtctctgttctctggcag
aggaggtcccttggcagcgggaagcgccctctctttctctcgccgccgc
tccgagtctgcgccctggtgccaggcgctcagctcggcgctcccctgtg
ctcgcccggcgcccactcattcgcagcccggccttcgtcgccgccgcct
ccctgctgctcctcctcctttccccagcccgccgcggccatggcggaca
gcgccagcgagagcgacacggacggggcggggggcaacagcagcagctc
ggccgccatgcagtcgtcctgctcgtcgacctcgggcggcggcggtggc
ggcgggggaggcggcggcggtgggaagtcggggggcattgtcatctcgc
cgttccgcctggaggagctcaccaaccgcctggcctcgctgcagcaaga
gaacaaggtgctgaagatagagctggagacctacaaactgaagtgcaag
gcactgcaggaggagaaccgcgacctgcgcaaagccagcgtgaccat
c-3′

The gene of interest of the second probe refers to the exon 12 of human RET gene. The sequence of exon 12 is shown in SEQ ID NO: 10:

5′-
gaggatccaaagtgggaattccctcggaagaacttggttcttggaaa
aactctaggagaaggcgaatttggaaaagtggtcaaggcaacggcct
tccatctgaaaggcagagcagggtacaccacggtggccgtgaagatg
ctgaaag-3′

The first probe and the second probe are respectively a linear, single-stranded DNA, having a structure of “first region-third region-second region”; where “-” represents direct ligation (via a phosphodiester bond) and/or via a linker (for example, several consecutive bases).

When the first probe and/or the second probe bind to the gene of interest, the first region and the second region are simultaneously folded toward the third region and bind to the single-stranded DNA of the gene of interest. At this time, there is no phosphodiester bond between the first region and the second region, and the first probe and/or the second probe comprises an open circular single-stranded DNA.

The first region of the first probe comprises 11-15 nucleotides (nt) and 60%-75% GC; the second region of the first probe comprises 14-18 nt and 60%-75% GC; and the temperature of melting (Tm) of the second region of the first probe is 3-15° C. higher than that of the first region of the first probe.

Tm is the temperature at which the absorbance of UV light is 50% between the maximum and minimum during the thermal denaturation of the double-stranded helix structure of DNA. The Tm and the number of the nucleotides of a probe are positively correlated with the GC contents. The Tm of the first probe is measured when the concentration of the first probe is 100 μM and the concentration of salt ions is 50 nM.

The first region of the first probe has 11 nt, 12 nt, 13 nt, 14 nt, or 15 nt, preferably 13 nt. The GC contents of the first region of the first probe is 60%, 66.7%, 69.2%, 73.3%, or 75%, preferably 69.2%.

The second region of the first probe has 14 nt, 15 nt, 16 nt, 17 nt, or 18 nt, preferably 16 nt. The GC contents of the second region of the first probe is 60%, 66.7%, 68.8%, 70.6%, or 75%, preferably 68.8%.

The Tm of the second region of the first probe is higher than that of the first region of the first probe by 11° C., 11.5° C., 12° C., 12.5° C., 13° C., 13.2° C., 13.5° C., 14° C., 14.5° C. or 15° C., preferably by 13.2° C.

The first region of the second probe comprises 12-15 nt and 60%-75% GC; the second region of the second probe comprises 20-24 nucleotides and 47.8%-55% GC; the Tm of the second region of the second probe is −3-15° C. high than that of the first region of the second probe.

The Tm of the second probe is measured when the concentration of the second probe is 100 μM and the concentration of the salt ions is 50 μM.

The first region of the second probe has 12 nt, 13 nt, 14 nt, or 15 nt, preferably 13 nt. The GC contents of the first region of the second probe is 60%, 64.3%, 69.2%, 75%, preferably 69.2%.

The second region of the second probe has 20 nt, 21 nt, 22 nt, 23 nt, or 24 nt, preferably 22 nt. The GC contents of the second region of the second probe is 47.8%, 52.4%, 50%, or 55%, preferably 50%.

The Tm of the second region of the second probe is higher than that of the first region of the second probe by 4° C., 4.5° C., 5° C., 5.5° C., 6° C., 6.5° C., 7° C., 7.5° C., 8° C., 8.5° C. ° C., 9° C., 9.2° C., 9.5° C., 9.8° C., 10° C., 10.3° C., 10.5° C., 10.8° C., 11° C., 11.3° C., 11.5° C. or 11° C., preferably by 10.5° C.

The Tm of the first region of the first probe is 46-50° C., preferably 47.1° C.; and the Tm of the second region of the first probe is 58-62° C., preferably 60.3° C.

The Tm of the first region of the first probe is 46° C., 46.5° C., 47° C., 47.1° C., 47.6° C., 48° C., 48.5° C., 49° C., 49.5° C., or 50° C., preferably 47.1° C.

The Tm of the second region of the first probe is 58° C., 58.5° C., 59° C., 59.5° C., 60° C., 60.3° C., 60.5° C., 61° C., 61.5° C., or 62° C., preferably 60.3° C.

The Tm of the second region of the first probe is 12-14° C. higher than that of the first region of the first probe, preferably 13.2° C. higher.

The first probe is used to recognize the DNA sequences that have been cleaved by the type II restriction endonuclease (preferably, the restriction enzymes are FspI, Cac8I or CdiI which recognize the palindrome sequence in the exon 1 of the CCDC6 gene) and by the exonuclease (preferably, Lambda exonuclease). The palindrome sequence recognized by FspI is: TGCCGA; the palindrome sequence recognized by restriction enzyme Cac8I is: GCNNGC; and the palindrome sequence recognized by restriction enzyme CdiI is: CATCG. More preferably, the restriction enzyme is FspI.

The Tm of the first region of the second probe is 52-54° C., preferably 53.1° C. The Tm of the second region of the second probe is 62-65° C., preferably 63.6° C.

The Tm of the first region of the second probe is 52° C., 52.3° C., 52.5° C., 52.8° C., 53° C., 53.1° C., 53.3° C., 53.5° C., 53.8° C. or 54° C., preferably 53.1° C.

The Tm of the second region of the second probe is 62° C., 62.5° C., 62.8° C., 63° C., 63.3° C., 63.6° C., 64° C., 64.2° C., 64.5° C. or 65° C., preferably 63.6° C.

The Tm of the second region of the second probe is 9-11° C. higher than that of the first region of the second probe, preferably 10.5° C. higher.

The second probe is used to recognize the DNA sequences that have been cleaved by the type II restriction endonuclease (preferably, restriction enzymes are RsaI, Hpyl8I, MslI or AleI which recognize the palindrome sequence in the exon 12 of the RET gene) and by the exonuclease (preferably Lambda Exonuclease). The palindrome sequence recognized by RsaI is: gtac; the palindrome sequence recognized by Hpyl8I is: gtnnac; the palindrome sequence recognized by MslI is: caynnnnrtg (SEQ ID NO: 21); and the palindrome sequence recognized by AleI is: cacnnnngtg (SEQ ID NO: 22). More preferably, the restriction enzyme is RsaI.

The first probe and/or the second probe is 80-90 nt in length; and more preferably, the third region of the first probe and/or the second probe is 40-55 nt in length.

The first probe is 80-90 nt in length, and the third region is 40-55 nt in length.

The second probe is 80-90 nt in length, and the third region is 40-55 nt in length.

By controlling the total number of nucleotides of each probe and the number of nucleotides in the third region, each probe can be combined with the target gene and form a loop. The looped probe is a linear self-replication probe with higher detection efficiency.

Since the fusion site of the CCDC6 gene and RTE gene is close to the 3′ end of exon 1 of the CCDC6 gene, the first probe is designed to be close to the 3′ end. In this way, the two fluorescent spots corresponding to the two genes that are physically far apart are close in distance, ensuring the accurate detection of the CCDC6-RET fusion gene.

First Probe

The first region of the first probe comprises a nucleotide sequence as follows:

1) the nucleotide sequence shown in SEQ ID NO: 1; or,

2) the complementary sequence or homologous sequence of the nucleotide sequence shown in SEQ ID NO: 1; or

3) a nucleotide sequence which is obtained by adding, deleting, or substituting one or more (for example, 1-3) bases to the nucleotide sequence shown in SEQ ID NO: 1 and is complementarily binds to the target gene; and/or

The second region of the first probe comprises a nucleotide sequence as follows:

1) the nucleotide sequence shown in SEQ ID NO: 2; or

2) the complementary sequence or homologous sequence (preferably a 90% similarity) of the nucleotide sequence shown in SEQ ID NO: 2; or

3) a nucleotide sequence which is obtained by adding, deleting, or substituting one or more (for example, 1-5) bases to the nucleotide sequence shown in SEQ ID NO: 2 and is complementarily binds to the target gene.

The homologous sequence of the first region of the first probe has at least 90% similarity with the nucleotide sequence shown in SEQ ID NO: 1.

The homologous sequence of the second region of the first probe has at least 90% similarity with the nucleotide sequence shown in SEQ ID NO: 2.

The nucleotide sequence of the first region of the first probe is shown in SEQ ID NO: 1: 5′-ctcctgcagtgcc-3′, and/or the nucleotide sequence of the second region of the first probe is shown in SEQ ID NO: 2: 5′-gcaggtcgcggttctc-3′.

The nucleotide sequence of the first region is complementary to the nucleotide sequence shown in SEQ ID NO: 1, and/or the nucleotide sequence of the second region is complementary to the nucleotide sequence shown in SEQ ID NO: 2.

The complementary nucleotide sequence hybridizes to the nucleotide sequence shown in SEQ ID NO: 1 and/or SEQ ID NO: 2 under stringent conditions. The “stringent conditions” refer to the conditions under which the probe hybridizes more strongly to the sequence of interest than to the other sequences. The strict hybridization conditions are sequence-dependent and differ with varying environmental parameters. Stringent hybridization and/or washing conditions can identify target sequences that are 100% complementary to the probe.

The nucleotide sequence of the first region is homologous to the nucleotide sequence shown in SEQ ID NO: 1, and/or the nucleotide sequence of the second region is homologous to the nucleotide sequence shown in SEQ ID NO: 2. The homologous sequence of the first region includes, but is not limited to, the nucleotide sequences with about 90%, 91%, 92%, 93%, 94%, 95%, 96%, 97%, 98%, or 99% similarity with the nucleotide sequence shown in SEQ ID NO: 1. The homologous sequence of the second region includes, but is not limited to, the nucleotide sequences with about 90%, 91%, 92%, 93%, 94%, 95%, 96%, 97%, 98%, or 99% similarity with the nucleotide sequence shown in SEQ ID NO: 2.

The nucleotide sequence of the first region (and/or the second region) is obtained by adding, deleting, or substituting one or more (for example, 1-3) bases to the nucleotide sequence shown in SEQ ID NO: 1 (and/or the nucleotide sequence shown in SEQ ID NO: 2) and is complementary to the gene of interest. Since the third region does not bind to the gene of interest, the binding of the first region to the gene of interest will not be affected when adding 1-3 (or more) bases, deleting 1-3 bases, or substituting 1-3 bases at the end of the first region close to the third region.

The first probe comprises a sequence as follows:

1) the nucleotide sequence shown in SEQ ID NO: 3; or,

2) the complementary sequence or homologous sequence of the nucleotide sequence shown in SEQ ID NO: 3 (preferably a 70% similarity);

3) a nucleotide sequence obtained by adding, deleting, or substituting one or more bases to the nucleotide sequence shown in SEQ ID NO: 3.

The nucleotide sequence of the first probe is shown in SEQ ID NO: 3:

5′-_

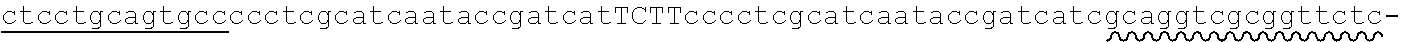

3′;

where the underlined straight line indicates the nucleotide sequence of the first region, the underlined wavy line indicates the nucleotide sequence of the second region, and the middle nucleotide sequence is the third region.

Since the third region does not bind to the gene of interest but binds to a first fluorescent probe to make the probe color, the sequence of the third region is arbitrarily variable.

The sequence of the third region shown in SEQ ID NO: 3 is variable.

The nucleotide sequence of the first probe of the disclosure is the complementary sequence of the nucleotide sequence shown in SEQ ID NO: 3. The complementary sequence is a nucleotide sequence that hybridizes with the nucleotide sequence of SEQ ID NO: 3 under stringent conditions.

The nucleotide sequence of the probe is a homologous sequence of the nucleotide sequence shown in SEQ ID NO: 3. The homologous sequence includes, but is not limited to, a nucleotide sequence with about 70%, 72%, 75%, 78%, 80%, 82%, 85%, 88%, 90%, 93%, 95%, 96%, 97%, 98%, or 99% similarity to the nucleotide sequence shown in SEQ ID NO: 3.

The nucleotide sequence of the probe is obtained by adding, deleting, or substituting one or more (for examples, 1-10, preferably 1-5, more preferably 1-3) bases in the nucleotide sequence shown in SEQ ID NO: 3.

In the third region, adding 1-10 or more bases, or deleting 1-10 bases, or substituting 1-10 bases has no effect on binding of the probe to the gene of interest.

The first fluorescent probe comprises a nucleotide sequence as follows:

1) the nucleotide sequence shown in SEQ ID NO: 4; or,

2) the complementary sequence or homologous sequence of the nucleotide sequence shown in SEQ ID NO: 4;

3) a nucleotide sequence obtained by adding, deleting, or substituting one or more (for examples, 1-10) nucleotides in the nucleotide sequence shown in SEQ ID NO: 4 and complementarily binds to the target gene;

The nucleotide sequence of the first fluorescent probe is shown in SEQ ID NO: 4: 5′-ccctcgcatcaataccgatcat-3′.

The nucleotide sequence of the first fluorescent probe is the complementary sequence of the nucleotide sequence shown in SEQ ID NO: 4. The complementary nucleotide sequence is capable of hybridizing with the nucleotide sequence shown in SEQ ID NO: 4 under stringent conditions.

The nucleotide sequence of the first fluorescent probe is a homologous sequence of the nucleotide sequence shown in SEQ ID NO: 4. The homologous sequence includes, but is not limited to, a nucleotide sequence with about 70%, 72%, 75%, 78%, 80%, 82%, 85%, 88%, or 90% similarity to the nucleotide sequence shown in SEQ ID NO: 4.

The nucleotide sequence of the first fluorescent probe is a nucleotide sequence that complementarily binds to the first probe after adding, deleting, or substituting one or more (for example, 1-10) nucleotides in the nucleotide sequence shown in SEQ ID NO: 4. For example, adding, deleting, or substituting 1, 2, 3, 4, 5, 6, 7, 8, 9, 10 or more bases of the sequence shown in SEQ ID NO: 4, and the resulting sequence is complementary to the first probe.

The nucleotide sequence of the first fluorescent probe in 1), 2), or 3) is connected with a fluorescent label. The fluorescent label includes, but are not limited to, Cy3, Cy5, 6-FAM, 6-TET, 5-FITC, 6-TRITC, 5-TAMRA, 6-TAMRA, and AMC.

The nucleotide sequence of the first probe shown in SEQ ID NO: 3 comprises two repetitive sequences in series, and the repetitive sequence is the same as the sequence of the fluorescent probe, and there are complementary sequences between the repetitive sequences. The sequence of one probe that binds to two fluorescent probes increases the amount of fluorescent signal, and the fluorescent signal can be observed. A supplementary sequence comprising several bases is connected between two repetitive sequence to separate the two repetitive sequences and increase the spatial distance. When the two repetitive sequences are both bound to the fluorescent probe, avoiding a large fluorescent group reduces the binding efficiency of the fluorescent probe and the two repetitive sequences.

Second Probe

The first region of the second probe comprises a nucleotide sequence as follows:

1) the nucleotide sequence shown in SEQ ID NO: 6; or,

2) the complementary sequence or homologous sequence of the nucleotide sequence shown in SEQ ID NO: 6; or

3) a nucleotide sequence which is obtained by adding, deleting, or substituting one or more bases to the nucleotide sequence shown in SEQ ID NO: 6 and complementarily binds to the target gene; preferably, the homologous sequence of the first region of the second probe has at least 90% similarity with the nucleotide sequence shown in SEQ ID NO: 6.

The nucleotide sequence of the first region of the second probe is shown in SEQ ID NO: 6: 5′-GGAAGGCCGTTGC-3′.

The nucleotide sequence of the first region of the second probe is complementary to the nucleotide sequence shown in SEQ ID NO: 6.

The nucleotide sequence of the first region of the second probe is obtained by adding, deleting, or substituting one or more (for examples, 1-3) bases in the nucleotide sequence shown in SEQ ID NO: 6 and is complementary to the gene of interest. Since the third region does not bind to the gene of interest, the binding of the first region to the gene of interest will not be affected when adding 1-3 (or more) bases, deleting 1-3 bases, or substituting 1-3 bases at the end of the first region close to the third region.

The second region of the second probe comprises a nucleotide sequence as follows:

1) The nucleotide sequence shown in SEQ ID NO: 7; or,

2) The complementary sequence or homologous sequence of the nucleotide sequence shown in SEQ ID NO: 7; or

3) a nucleotide sequence which is obtained by adding, deleting, or substituting one or more bases to the nucleotide sequence shown in SEQ ID NO: 7 and complementarily binds to the target gene; preferably, the homologous sequence of the second region of the second probe has at least 90% similarity with the nucleotide sequence shown in SEQ ID NO: 7.

The nucleotide sequence of the second region of the second probe is shown in SEQ ID NO: 7: 5′-ACCCTGCTCTGCCTTTCAGAT-3′.

The nucleotide sequence of the second region of the second probe is complementary to the nucleotide sequence shown in SEQ ID NO: 7.

The nucleotide sequence of the second region of the second probe is homologous to the nucleotide sequence shown in SEQ ID NO: 7.

The nucleotide sequence of the second region is obtained by adding, deleting, or substituting one or more (for example, 1-5) bases to the nucleotide sequence shown in SEQ ID NO: 7 and is complementary to the gene of interest. Since the third region does not bind to the gene of interest, the binding of the second region to the gene of interest will not be affected when adding 1-5 (or more) bases, deleting 1-5 bases, or substituting 1-5 bases at the end of the second region close to the third region.

The second probe comprises a sequence as follows:

1) the nucleotide sequence shown in SEQ ID NO: 8; or,

2) the complementary sequence or homologous sequence of the nucleotide sequence shown in SEQ ID NO: 8; or

3) a nucleotide sequence obtained by adding, deleting, or substituting one or more bases to the nucleotide sequence shown in SEQ ID NO: 8.

The homologous sequence of the second region of the second probe has at least 70% similarity with the nucleotide sequence shown in SEQ ID NO: 8.

The nucleotide sequence of the second probe is shown in SEQ ID NO: 8:

GGAAGGCCGTTGCCTGCGAATAGCCATCCACTCCATTCTTCTGCGAATAGC

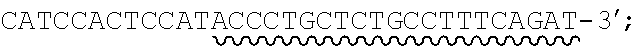

where the underlined straight line indicates the nucleotide sequence of the first region, the underlined wavy line indicates the nucleotide sequence of the second region, and the middle nucleotide sequence is the third region.

Since the third region does not bind to the gene of interest but binds to a second fluorescent probe to make the probe color, the sequence of the third region is arbitrarily variable.

The sequence of third region shown in SEQ ID NO: 8 is variable.

The nucleotide sequence of the second probe is the complementary sequence of the nucleotide sequence shown in SEQ ID NO: 8.

The nucleotide sequence of the second probe is a homologous sequence of the nucleotide sequence shown in SEQ ID NO: 8.

The nucleotide sequence of the second probe is obtained by adding, deleting, or substituting one or more (for examples, 1-10, preferably 1-5, more preferably 1-3) bases in the nucleotide sequence shown in SEQ ID NO: 8.

The second fluorescent probe comprises a sequence as follows:

1) The nucleotide sequence shown in SEQ ID NO: 9; or,

2) The complementary sequence or homologous sequence of the nucleotide sequence shown in SEQ ID NO: 9;

3) a nucleotide sequence in which the nucleotide sequence shown in SEQ ID NO: 9 is added, deleted, or replaced with one or more (for examples, 1-10) bases and complementarily binds to the second probe.

The nucleotide sequence of the second fluorescent probe is shown in SEQ ID NO: 9: 5′-ccctcgcatcaataccgatcat-3′.

The nucleotide sequence of the second fluorescent probe is complementary to the nucleotide sequence shown in SEQ ID NO: 9.

The nucleotide sequence of the second fluorescent probe is homologous to the nucleotide sequence shown in SEQ ID NO: 9.

The nucleotide sequence of the second fluorescent probe is obtained by adding, deleting, or substituting one or more (for example, 1-10) bases to the nucleotide sequence shown in SEQ ID NO: 9 and is complementary to the second probe. Addition of 1-10 or more bases, or deletion of 1-10 bases, or replacement of 1-10 bases has no effect on binding of the probe to the gene of interest.

The nucleotide sequence of the second fluorescent probe in 1), 2), or 3) is connected with a fluorescent label. The fluorescent label includes, but are not limited to, Cy3, Cy5, 6-FAM, 6-TET, 5-FITC, 6-TRITC, 5-TAMRA, 6-TAMRA, and AMC.

The nucleotide sequence of the second probe shown in shown SEQ ID NO: 8 also comprises two repeated sequences in series. The repeated sequence is the same as that of the repeated sequences and there are complementary sequences between the repetitive sequences.

The disclosure further provides a kit for in situ detection of human CCDC6-RET fusion gene, the kit comprising the first probe and the second probe.

The kit further comprises a cell permeation system, a blunt end system, a target nucleotide exposure system, a probe locking system, a signal amplification system, a signal detection system, or a combination thereof. The kit optionally comprises a cleaning system.

Cell Permeation System

The cell permeation system contains proteinase K (with a concentration of 5 mg/mL-30 mg/mL), Tris-HCl buffer, EDTA, and SDS. The cell permeation system is capable of permeating the cell membrane and nuclear membrane, allowing the reagents to react in the nucleus.

Blunt End System

The blunt end system contains endonuclease FspI (can be replaced with Cac8I, or CdiI) for cleaving the exon 1 of the CCDC6 gene; endonuclease RsaI for cleaving the exon 12 of the RET gene (can be replaced by Hpyl8I, MslI, or AleI), CutSmart buffer, and nuclease-free ultrapure water. The blunt end system cleaves the palindrome sequence near the target DNA site that have bound to the probe, producing blunt end fragment.

Target Nucleotide Exposure System

The target nucleotide exposure system contains Lambda Exonuclease, Exonuclease buffer, and nuclease-free ultrapure water. The target nucleotide exposure system degrades the single-stranded DNA in 3′ to 5′ direction from the blunt end, so that the target single-stranded DNA that have bound to the probe is exposed.

Probe Locking System

The probe locking system contains the first probe, the second probe, DNA Ligase buffer, ATP and nuclease-free ultrapure water. In the probe locking system, the first probe and the second probe bind to their respective target single-stranded DNAs, and are circularized at the binding site by ligase to form closed circular single-stranded DNAs.

Signal Amplification System

The signal amplification system contains DNA polymerase, DNA polymerase buffer, dNTPs, DTT and nuclease-free ultrapure water. The closed circular single-stranded DNAs perform linear self-replication in the presence of DNA polymerase, thereby producing a large number of circular single-stranded probes comprising repetitive sequences.

Signal Detection System

The signal detection system contains the first fluorescent probe, the second fluorescent probe, formamide, sodium chloride, sodium citrate, salmon sperm DNA and nuclease-free ultrapure water. In the signal detection system, the first fluorescent probe and the second fluorescent probe are respectively bound to the circular single-stranded probes comprising repetitive sequences, thereby showing the localization of the first probe and the second probe in the nucleus.

Cleaning System

The cleaning system contains Tris-HCl, NaCl, Tween20 and nuclease-free ultrapure water. The cleaning system is used as a cleaning liquid that washes the reaction liquid after each step of the reaction is complete.

The disclosure further provides a method for in situ detection of human CCDC6-RET fusion gene.

The method comprises the following steps:

(a) fixing a cell sample to be tested, treating the fixed cell sample with the cell permeation system, and optionally washing the permeated cell sample with the cleaning system;

(b) treating the cell sample obtained in (a) with the blunt end system, and optionally washing the permeated cell sample with the cleaning system;

(c) treating the cell sample obtained in (b) with the target nucleotide exposure system, and optionally washing the permeated cell sample with the cleaning system;

(d) treating the cell sample obtained in (c) with the probe locking system, optionally washing the permeated cell sample with the cleaning system, and optionally drying the cell sample;

(e) treating the cell sample obtained in (d) with the signal amplification system, and optionally washing the permeated cell sample with the cleaning system;

(f) treating the cell sample obtained in (d) with the signal detection system, and optionally washing the permeated cell sample with the cleaning system, and optionally drying the cell sample; and

(g) sealing the cell sample, and observing the color of the fluorescent.

Referring to FIG. 1, the working principle of the method for in situ detection of the CCDC6-RET fusion gene is as follows:

The cell nuclear membrane is punched with proteinase K, and the palindrome sequence is cleaved near the target site bound by the probe sequence through a type II restriction endonuclease, exposing blunt ends. The exonuclease degrades one strand of the double-stranded DNA in the 5′->3′ direction from the blunt end, and the target single-stranded DNA bound by the probe is exposed. The first probe and the second probe are respectively circularized at the binding site in the presence of ligase to form closed circular single-stranded DNA. The closed circular single-stranded DNA performs linear self-replication in the presence of DNA polymerase, and the generated sequence contains a large number of repetitive sequences that are not found in human genes. Specific fluorescent probe binds with the repetitive sequences and display the location of the first probe and the second probe, thereby detecting the location of the CCDC6-RET fusion gene in the nucleus.

The following advantages are associated with the probes, kits and methods of the disclosure:

1. In combination with the in-situ detection method, the disclosure uses the specific first probe and the second probe to amplify the target signal. The fluorescent sites of the two probes are judged by whether they are close, and the location and copy number of the CCDC6-RET fusion gene in the nucleus of the cells or clinical tissue samples are observed visually.

2. The probes or the kit of the disclosure can be used for the detection of mutations of human CCDC6-RET fusion gene in the nucleus in solid tumors, and a small amount of cells or clinical tissue samples is consumed, which has a wide range of applicability.

3. The method of the disclosure does not involve nucleic acid extraction and digital signal conversion, and has the advantages of lower cost, higher sensitivity, better specificity, and simpler operation over the prior arts.

### BRIEF DESCRIPTION OF THE DRAWINGS

FIG. 1 is the working principle of the method of the disclosure.

FIG. 2 is a diagram showing the results of detecting fusion genes in in vitro cell lines in accordance with one embodiment of the disclosure.

FIG. 3 is a diagram showing the results of detecting fusion genes using paraffin tissue sections in accordance with one embodiment of the disclosure.

FIGS. 4A to 4D are diagrams showing the results of detecting fusion gene positive single cells using paraffin tissue sections in accordance with one embodiment of the disclosure.

FIGS. 5A to 5D are diagrams showing the results of detecting fusion gene negative single cells using paraffin tissue sections in accordance with one embodiment of the disclosure.

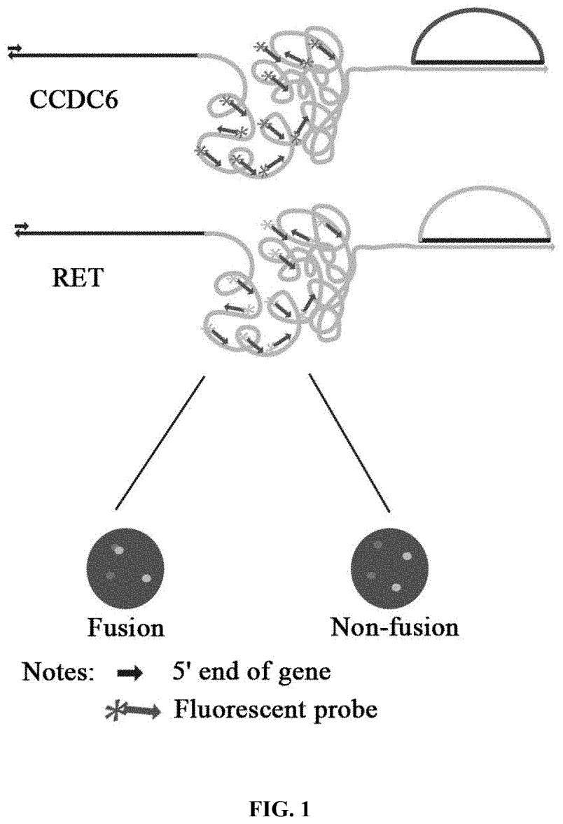

### DETAILED DESCRIPTION

To further illustrate the disclosure, embodiments detailing probes, kit, and method of the disclosure are described below. It should be noted that the following embodiments are intended to describe and not to limit the disclosure.

The materials and reagents used in the following examples can be obtained from commercial sources unless otherwise specified.

1×CutSmart buffer contains 50 mM/L potassium acetate, 20 mM/L Tri-acetate, 10 mM/L magnesium acetate, and 0.1 mg/mL BSA.

1×Exonuclease buffer contains 50 mM/L potassium acetate, 20 mM/L Tri-acetate, 10 mM/L magnesium acetate, and 0.1 mg/mL BSA.

1×DNA Ligase buffer contains 40 mM/L Tris-HCl, 10 mM/L magnesium chloride, 10 mM/L DTT, 0.5 mM/L ATP, and 0.05 Weiss U/μL DNA.

1×DNA polymerase buffer contains 33 mM/L Tris-acetic acid, 10 mM/L magnesium acetate, 66 mM/L potassium acetate, and 0.1% (v/v) Tween20.

### Example 1-6

The first probe in Examples 1-6 is shown in Table 1 and the second probe is shown in Table 2.

TABLE 1
First probe

| | | First region | | | Second region | | |
|---|---|---|---|---|---|---|---|
| | Nucleotide | Number | | | Number | | |
| Group | sequence | of bases | GC | Tm | of bases | GC | Tm |
| Example 1 | SEQ ID NO: 3 | 13 | 69.2% | 47.1° C. | 16 | 68.8% | 60.3° C. |
| Example 2 | SEQ ID NO: 11 | 15 | 66.7% | 54.9° C. | 15 | 66.7% | 58° C. |
| Example 3 | SEQ ID NO: 12 | 15 | 60% | 51.9° C. | 16 | 68.8% | 60.3° C. |
| Example 4 | SEQ ID NO: 13 | 15 | 60% | 54.2° C. | 17 | 70.6% | 64.3° C. |
| Example 5 | SEQ ID NO: 14 | 19 | 57.9% | 62.3° C. | 11 | 81.8% | 49.4° C. |
| Example 6 | SEQ ID NO: 15 | 14 | 57.1% | 50° C. | 17 | 70.6% | 63.4° C. |

TABLE 2
Second probe

| | | First region | | | Second region | | |
|---|---|---|---|---|---|---|---|
| | Nucleotide | Number | | | Number | | |
| Group | sequence | of bases | GC | Tm | of bases | GC | Tm |
| Example 1 | SEQ ID NO: 8 | 13 | 69.23% | 53.1° C. | 22 | 50% | 63.6° C. |
| Example 2 | SEQ ID NO: 16 | 15 | 60% | 55.9° C. | 15 | 60% | 53.2° C. |
| Example 3 | SEQ ID NO: 17 | 12 | 66.7% | 46.7° C. | 18 | 55.56% | 60.7° C. |
| Example 4 | SEQ ID NO: 18 | 14 | 57.14% | 51.2° C. | 18 | 55.56% | 60.7° C. |
| Example 5 | SEQ ID NO: 19 | 18 | 50% | 60.4° C. | 14 | 64.29% | 48.8° C. |
| Example 6 | SEQ ID NO: 20 | 12 | 58.33% | 46.9° C. | 21 | 52.38% | 63.4° C. |

### Example 7

A kit for detecting CCDC6-RET fusion gene comprises:

(1) Cell permeation system: 20 mg/mL proteinase K, Tris-HCl buffer, EDTA, SDS;

(2) Blunt end system: 0.5 U/μL RsaI endonuclease, 1×CutSmart buffer, nuclease-free ultrapure water;

(4) Target nucleotide exposure system: 0.4 U/μL Lambda exonuclease, 1×Exonuclease buffer, nuclease-free ultrapure water;

(5) Probe locking system: specific probes (the first probe and the second probe) having a final concentration of 100 μM/L, 0.05 Weiss U/μL DNA ligase, 1×DNA Ligase buffer, 0.5 mM/L ATP, and nuclease-free ultrapure water;

(6) Signal amplification system: final concentration of 1 U/μL DNA polymerase, 1×DNA polymerase buffer, 2.5 mM/L dNTPs, 1 mM/L DTT, and nuclease-free ultrapure water;

(7) Signal detection system: the first fluorescent probe, the second fluorescent probe, 20% (v/v) formamide, 0.3 M/L sodium chloride, 0.03 M/L sodium citrate, 0.5 μg/μL salmon sperm DNA, and nuclease-free ultrapure water.

(8) Cleaning system: 0.1 M/L Tris-HCl, 0.15 M/L NaCl, 0.05% (v/v) Tween20, and nuclease-free ultrapure water.

### Example 8

A method for detecting CCDC6-RET fusion gene using the kit in Example 7 comprises:

(1) cell samples were fixed in vitro and treated with the cell permeation system at 37° C. for 3-4 minutes; the resulting liquid was discarded and the ultrapure water was added to the resulting product; the mixture was washed with 70%, 85%, and 100% ethanol in turn and dried;

(2) the cell samples obtained in (1) were treated with the blunt end system at 37° C. for 1 hour; the resulting liquid was discarded; the resulting product was treated with the cleaning system; and the resulting liquid was discarded;

(3) the cell samples obtained in (2) were treated with the target nucleotide exposure system at 37° C. for 0.5 hours; the resulting liquid was discarded; the resulting product was treated with the cleaning system;

(4) the cell samples obtained in (3) were treated with the probe locking system at 37° C. for 0.5 hours; the resulting liquid was discarded; the resulting product was treated with the cleaning system; the resulting liquid was discarded; and the mixture was washed with 70%, 85%, and 100% ethanol in turn and dried;

(5) the cell samples obtained in (4) were treated with the signal amplification system; the resulting liquid was discarded; the resulting product was treated with the cleaning system; and the resulting liquid was discarded;

(6) the cell samples obtained in (5) were treated with the signal detection system; he resulting liquid was discarded; the resulting product was treated with the cleaning system; the resulting liquid was discarded; and the mixture was washed with 70%, 85%, and 100% ethanol in turn and dried;

(7) a mounting medium with ADPI were added to the cell samples obtain in (6) and the mounted cell samples was sealed; and

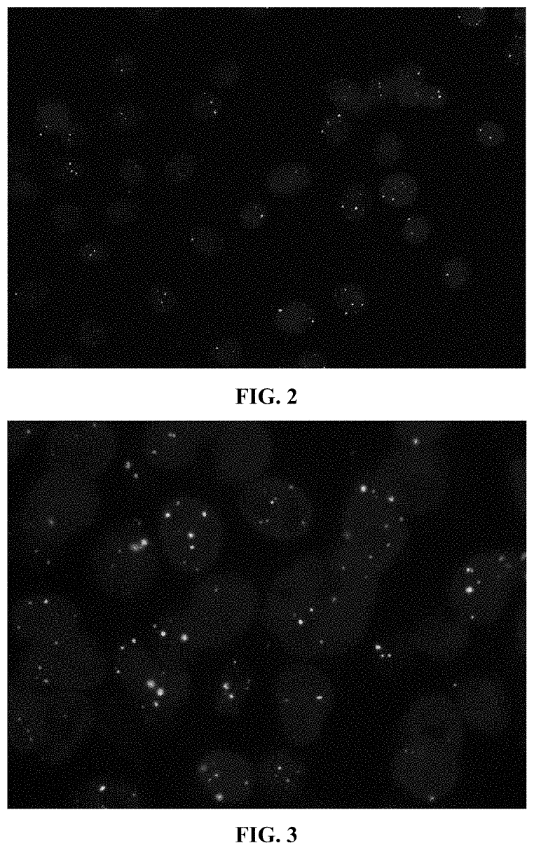

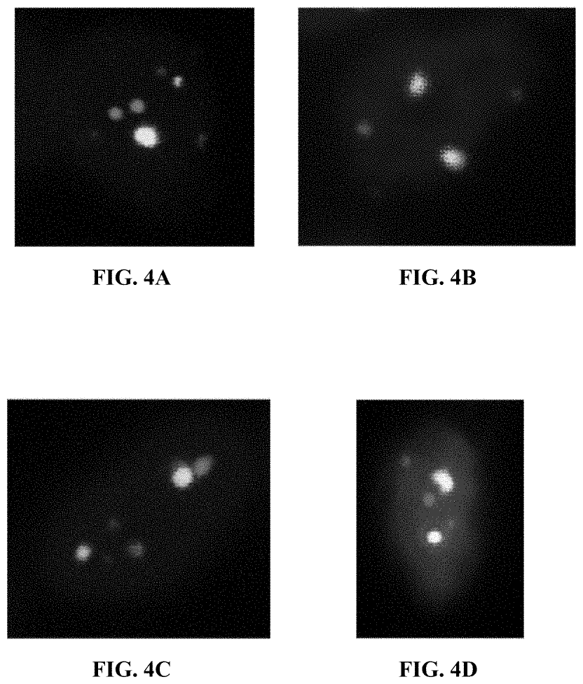

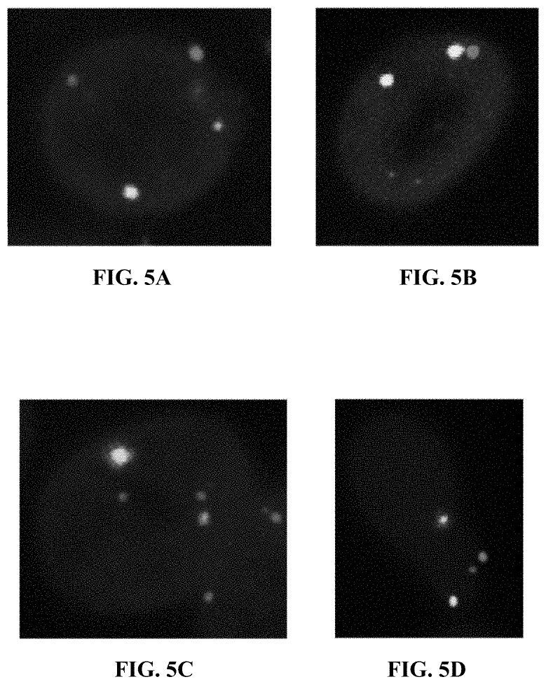

(8) the color of the fluorescent was observed under a fluorescence microscope and the results were shown in FIG. 2;

### Example 9

A method for detecting CCDC6-RET fusion gene using the kit in Example 7 comprises:

(1) cell samples were embedded into paraffin blocks, cut into sections, and treated with the cell permeation system at 37° C. for 15-20 minutes; the resulting liquid was discarded and the ultrapure water was added to the resulting product; the mixture was washed with 70%, 85%, and 100% ethanol in turn and dried;

(2) the cell samples obtained in (1) were treated with the blunt end system at 37° C. for 1 hour; the resulting liquid was discarded; the resulting product was treated with the cleaning system; and the resulting liquid was discarded;

(3) the cell samples obtained in (2) were treated with the target nucleotide exposure system at 37° C. for 0.5 hours; the resulting liquid was discarded; the resulting product was treated with the cleaning system;

(4) the cell samples obtained in (3) were treated with the probe locking system at 37° C. for 0.5 hours; the resulting liquid was discarded; the resulting product was treated with the cleaning system; the resulting liquid was discarded; and the mixture was washed with 70%, 85%, and 100% ethanol in turn and dried;

(5) the cell samples obtained in (4) were treated with the signal amplification system at 44° C. for 1 hour; the resulting liquid was discarded; the resulting product was treated with the cleaning system; and the resulting liquid was discarded;

(6) the cell samples obtained in (5) were treated with the signal detection system at 37° C. for 10 minutes; the resulting liquid was discarded; the resulting product was treated with the cleaning system; the resulting liquid was discarded; and the mixture was washed with 70%, 85%, and 100% ethanol in turn and dried;

(7) a mounting medium with ADPI were added to the cell samples obtain in (6) and the mounted cell samples was sealed; and

(8) the color of the fluorescent was observed under a fluorescence microscope and the results were shown in FIG. 3;

It will be obvious to those skilled in the art that changes and modifications may be made, and therefore, the aim in the appended claims is to cover all such changes and modifications.

What is claimed is:

1. A pair of probes for identifying CCDC6-RET fusion gene in a cell nucleus, the pair of probes comprising:
1) a first probe for identifying a CCDC6 gene; and
2) a second probe for identifying a RET gene.

2. The pair of probes of claim 1, wherein the first probe is configured to identify an exon 1 of the CCDC6 gene, and/or the second probe is configured to identify an exon 12 of the RET gene.

3. The pair of probes of claim 1, wherein:
the first probe and the second probe each comprise: (1) a first region complementary to a 5′-end sequence of a gene of interest; (2) a second region complementary to a 3′-end sequence of the gene of interest; and (3) a third region which is a circular sequence complementary to a fluorescent probe and located between the first region and the second region;
the first region of the first probe comprises 11-15 nucleotides (nt) and 60%-75% GC; the second region of the first probe comprises 14-18 nt and 60%-75% GC; and a temperature of melting (Tm) of the second region of the first probe is 3-15° C. higher than that of the first region of the first probe; the Tm of the first region of the first probe is higher than 45° C.; and/or
the first region of the second probe comprises 12-15 nucleotides and 60%-75% GC; the second region of the second probe comprises 20-24 nucleotides and 47.8%-55% GC; the Tm of the second region of the second probe is −3-15° C. high than that of the first region of the second probe; and the Tm of the first region of the second probe is higher than 45° C.

4. The pair of probes of claim 3, wherein:
the Tm of the first region of the first probe is 46-50° C., and the Tm of the second region of the first probe is 58-62° C.; the Tm of the second region of the first probe is 12-14° C. higher than that of the first region of the first probe;
the Tm of the first region of the second probe is 52-54° C.; the Tm of the second region of the second probe is 62-65° C.; the Tm of the second region of the second probe is 9-11° C. higher than that of the first region of the second probe; and
the first probe and/or the second probe comprises 80-90 nt.

5. The pair of probes of claim 4, wherein:
the Tm of the first region of the first probe is 47.1° C., and the Tm of the second region of the first probe is 60.3° C.;
the Tm of the first region of the second probe is 53.1° C.; the Tm of the second region of the second probe is 63.6° C.; and
the first probe and/or the second probe comprises 40-55 nt.

6. The pair of probes of claim 3, wherein:
the first region of the first probe comprises a nucleotide sequence as follows:
a nucleotide sequence shown in SEQ ID NO: 1; or
a complementary sequence or homologous sequence of the nucleotide sequence shown in SEQ ID NO: 1; or
a nucleotide sequence which is obtained by adding, deleting, or substituting one or more bases to the nucleotide sequence shown in SEQ ID NO: 1 and is complementarily binds to a target gene; and/or
the second region of the first probe comprises a nucleotide sequence as follows:
a nucleotide sequence shown in SEQ ID NO: 2; or
a complementary sequence or homologous sequence of the nucleotide sequence shown in SEQ ID NO: 2; or
a nucleotide sequence which is obtained by adding, deleting, or substituting one or more bases to the nucleotide sequence shown in SEQ ID NO: 2 and is complementarily binds to the target gene.

7. The pair of probes of claim 3, wherein:
the first region of the second probe comprises a nucleotide sequence as follows:
a nucleotide sequence shown in SEQ ID NO: 6; or,
a complementary sequence or homologous sequence of the nucleotide sequence shown in SEQ ID NO: 6; or
a nucleotide sequence which is obtained by adding, deleting, or substituting one or more bases to the nucleotide sequence shown in SEQ ID NO: 6 and complementarily binds to a target gene;
the second region of the second probe comprises a nucleotide sequence as follows:
a nucleotide sequence shown in SEQ ID NO: 7; or,
a complementary sequence or homologous sequence of the nucleotide sequence shown in SEQ ID NO: 7; or
a nucleotide sequence which is obtained by adding, deleting, or substituting one or more bases to the nucleotide sequence shown in SEQ ID NO: 7 and complementarily binds to the target gene.

8. The pair of probes of claim 3, wherein:
the first probe comprises a sequence as follows:
a nucleotide sequence shown in SEQ ID NO: 3; or,
a complementary sequence or homologous sequence of the nucleotide sequence shown in SEQ ID NO: 3; or
a nucleotide sequence obtained by adding, deleting, or substituting one or more bases to the nucleotide sequence shown in SEQ ID NO: 3;
the second probe comprises a sequence as follows:
a nucleotide sequence shown in SEQ ID NO: 8; or,
a complementary sequence or homologous sequence of the nucleotide sequence shown in SEQ ID NO: 8; or
a nucleotide sequence obtained by adding, deleting, or substituting one or more bases to the nucleotide sequence shown in SEQ ID NO: 8.

9. A kit for in situ detection of human CCDC6-RET fusion gene, the kit comprising the pair of probes of claim 1, and further comprising a cell permeation system, a blunt end system, a target nucleotide exposure system, a probe locking system, a signal amplification system, a signal detection system, or a combination thereof.

10. The kit of claim 9, wherein the kit further comprises a cleaning system.

11. A method for in situ detection of human CCDC6-RET fusion gene using the kit of claim 10, the method comprising:
a) fixing a cell sample to be tested, treating the fixed cell sample with the cell permeation system, and washing the permeated cell sample with the cleaning system;
b) treating the cell sample obtained in a) with the blunt end system, and washing the permeated cell sample with the cleaning system;
c) treating the cell sample obtained in b) with the target nucleotide exposure system, and washing the permeated cell sample with the cleaning system;
d) treating the cell sample obtained in c) with the probe locking system, washing the permeated cell sample with the cleaning system, and drying the cell sample;
e) treating the cell sample obtained in d) with the signal amplification system, and washing the permeated cell sample with the cleaning system;
f) treating the cell sample obtained in d) with the signal detection system, and washing the permeated cell sample with the cleaning system, and drying the cell sample; and
g) sealing the cell sample, and observing a color of the fluorescent of the probes.
